(19) United States
(12) Patent Application Publication
(10) Pub. No.: US 2025/0325575 A2
(43) Pub. Date: Oct. 23, 2025

(54) SYNTHESIS OF 3 -RNA OLIGONUCLEOTIDES

(71) Applicant: ALNYLAM PHARMACEUTICALS, INC., Cambridge, MA (US)

(72) Inventors: Jayaprakash K. NAIR, Cambridge, MA (US); Juan C. SALINAS, Cambridge, MA (US); John Frederick BRIONES, Cambridge, MA (US); Mark K. SCHLEGEL, Cambridge, MA (US); Shigeo MATSUDA, Cambridge, MA (US); Alexander V. KEL’IN, Cambridge, MA (US); Ligang ZHANG, Cambridge, MA (US); Martin A. MAIER, Cambridge, MA (US)

(73) Assignee: ALNYLAM PHARMACEUTICALS, INC., Cambridge, MA (US)

(21) Appl. No.: 17/779,706

(22) PCT Filed: Nov. 23, 2020
(86) PCT No.: PCT/US2020/061755

§ 371 Date: May 25, 2022

Related U.S. Application Data
(60) Provisional application No. 62/941,153, filed on Nov. 27, 2019.

(51) Int. Cl. A61K 31/704; A61K 9/00; A61K 9/127; A61K 47/24; A61K 47/26; A61P 35/00

(52) U.S. Cl. CPC A61K 31/704; A61K 9/0019; A61K 9/127; A61K 47/24; A61K 47/26; A61P 35/00

(57) ABSTRACT

The disclosure is directed to monomers and methods for synthesizing oligonucleotides comprising at least one nucleoside comprising a 3′-hydroxyl group.

### CROSS-REFERENCE TO RELATED APPLICATIONS

This application claims benefit under 35 U.S.C. § 119(e) of U.S. Provisional Application No. 62/941,153 filed Nov. 27, 2019, the content of which is incorporated herein by reference in its entirety.

### FIELD OF THE INVENTION

The invention relates to generally to nucleic acid chemistry and to the chemical synthesis of oligonucleotides. More particularly, the invention relates to monomers and methods for synthesizing oligonucleotides comprising at least one nucleoside comprising a 3′-hydroxyl group.

### BACKGROUND

Modified oligonucleotides are of great value in molecular biological research and in therapeutic applications. While, chemical synthesis of modified oligonucleotides is routine, ease and yield of many modified oligonucleotides is low. For example, commonly used protecting groups are unstable to conditions employed for deprotecting chemically synthesized oligonucleotides. This is especially problematic when preparing oligonucleotides comprising at least one nucleoside comprising a 3′-hydroxyl group. Thus, there remains a need in the art for monomers and methods for preparing such oligonucleotides. The present disclosure addresses, at least partially, this need.

### SUMMARY

The disclosure provides monomers and methods for preparing oligonucleotides with improved yields and lower impurities where the oligonucleotide has at least one, e.g., two, three, four or more nucleosides with a 3′-hydroxyl group. Generally, the method comprises coupling a free hydroxyl group on a nucleoside or oligonucleotide with a nucleoside phosphoramidite monomer having a triisopropylsilylether (TIPS) protected 3′-hydroxyl group. The coupling forms a phosphite triester intermediate which can be oxidized or sulfurized to form a phosphate triester or phosphorothioate intermediate.

Oligonucleotides having a predetermined length and sequence can be prepared by the method. For example, the oligonucleotides comprising from about 6 to about 50 nucleotides can be prepared using the method and monomers described herein. In some embodiments, the oligonucleotide comprises from about 10 to about 30 nucleotides.

In another aspect, the disclosure provides monomers, e.g., nucleoside phosphoramidite monomers having a triisopropylsilylether protected 3′-hydroxyl group. Generally, the monomer is of Formula (I):

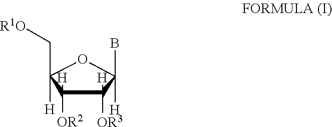

In Formula (I), B is a modified or unmodified nucleobase; R¹ is an acid labile hydroxyl protecting group; R² is —Si(R⁴)₃; R³ is —P(NR⁵R⁶)OR⁷; each R⁴ is independently optionally substituted alkyl, aryl, aralkyl, alkaryl, cycloalkyl, alkenyl, cycloalkenyl, alkynyl or cycloalkynyl; R⁵ and R⁶ are independently optionally substituted alkyl, aryl, aralkyl, alkaryl, cycloalkyl, alkenyl, cycloalkenyl, alkynyl or cycloalkynyl, or wherein R⁵ and R⁶ are linked to form a heterocyclyl; and R⁷ is optionally substituted alkyl, aryl, aralkyl, alkaryl, cycloalkyl, alkenyl, cycloalkenyl, alkynyl or cycloalkynyl.

In some monomers of Formula (I), B is adenine, guanine, cytosine or uracil; R¹ is dimethoxytrityl; R⁴, R⁵ and R⁶ are isopropyl; and R⁷ is β-cyanoethyl.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 is an HPLC trace of sequence 1 (aUfcaaAf(U-2′-OTBS)CfAfcuuuAfuUfgaguuuc, SEQ ID NO: 1) having U-2′-OTBS at N17 position after deprotection with ammonium hydroxide in ethanol showing the generation of FLP-2′-OTBS, FLP-OH and the cleaved (16mer)

FIG. 2 is an PLC trace of sequence 2 (aUfcaaAf(U-3′-OTBS)CfAfcuuuAfuUfgaguuuc, SEQ ID NO: 2) having U-3′-OTBS at N17 position after deprotection with ammonium hydroxide in ethanol showing the generation of FLP-3′-OTBS, FLP-OH and the cleaved (16mer)

FIG. 3 is an HPLC trace of sequence 3 (aUfcaaAf(G-3′-OTBS)CfAfcuuuAfuUfgaguuuc, SEQ ID NO: 3) having G-3′-OTBS at N17 position after deprotection with ammonium hydroxide in ethanol showing the generation of FLP-3′-OTBS, FLP-OH and the cleaved (16mer)

FIG. 4 is an HPLC trace of sequence 4 (aUfcaaAf(U-2′-OTOM)CfAfcuuuAfuUfgaguuuc, SEQ ID NO: 4) having U-2′-OTOM at N17 position after deprotection with ammonium hydroxide in ethanol showing the generation of FLP-2′-OTOM, FLP-OH and the cleaved (16mer)

FIG. 5 is an HPLC trace of sequence 5 (aUfcaaAf(U-3′-OTOM)CfAfcuuuAfuUfgaguuuc, SEQ ID NO: 5) having U-3′-OTOM at N17 position after deprotection with ammonium hydroxide in ethanol showing the generation of FLP-3′-OTOM, FLP-OH and the cleaved (16mer).

FIG. 6 is an HPLC trace of sequence 6 (aUfcaaAf(U-3′-OTIPS)CfAfcuuuAfuUfgaguuuc, SEQ ID NO: 6) having U-3′-OTIPS at N17 position after deprotection with ammonium hydroxide in ethanol showing the generation of FLP-3′-OTIPS, FLP-OH and the cleaved (16mer).

FIG. 7 is an HPLC trace of sequence 6 (aUfcaaAf(U-3′-OTIPS)CfAfcuuuAfuUfgaguuuc, SEQ ID NO: 6) having U-3′-OTIPS at N17 position after deprotection with ammonium hydroxide in ethanol and HF/pyridine showing the generation of FLP-OH. 3′-OTPS protecting group in RNA can be effectively cleaved using HF/Pyridine treatment.

FIG. 8 shows deconvoluted mass spectrum of sequence 8 (asCfsguuu(U2p)caaagcAfcUfuuauusgsa, SEQ ID NO: 8) deprotected with conc. aqueous ammonium hydroxide at room temperature overnight. The major peaks correspond to the desired FLP (sequence 8) and the 3′-fragment (sequence 9 (caaagcAfcUfuuauusgsa, SEQ ID NO: 9)). Approximately 14% of the FLP still maintains a single N-2-isobutyryl protecting group (M=7663).

FIG. 9 shows deconvoluted mass spectrum 8 of sequence (asCfsguuu(U2p)caaagcAfcUfuuauusgsa, SEQ ID NO: 8) deprotected with conc. aqueous methylamine for 2 hours at room temperature overnight. The major peaks correspond to the desired FLP (sequence 8) and the 3′-fragment (sequence 9 (caaagcAfcUfuuauusgsa, SEQ ID NO: 9)).

FIG. 10 shows deconvoluted mass spectrum of sequence 8 (asCfsguuu(U2p)caaagcAfcUfuuauusgsa, SEQ ID NO: 8) deprotected with conc. aqueous methylamine for at room temperature overnight. The major peaks correspond to the desired FLP (sequence 8), the 3′-fragment (sequence 9 (caaagcAfcUfuuauusgsa, SEQ ID NO: 9)), and the 5′-fragment (sequence 10, asCfsguuu(U2p) P, SEQ ID NO: 10)).

FIG. 11 shows structures of some exemplary 3′-triisopropylsilyl ether (3′-TIPS) nucleoside monomers.

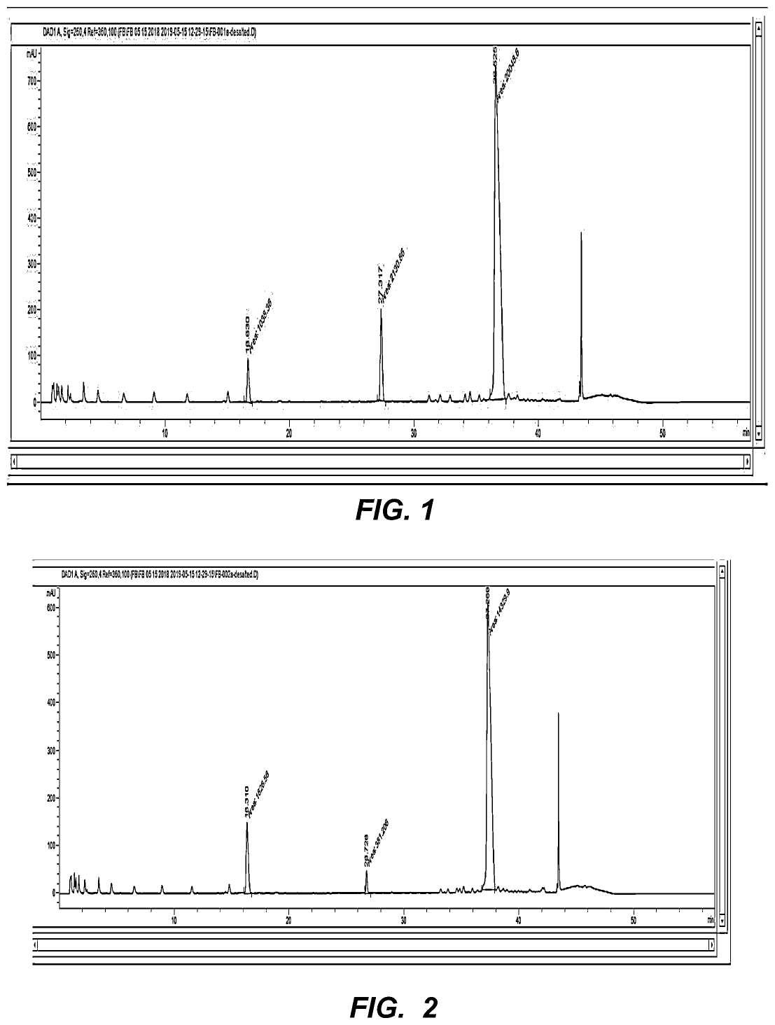

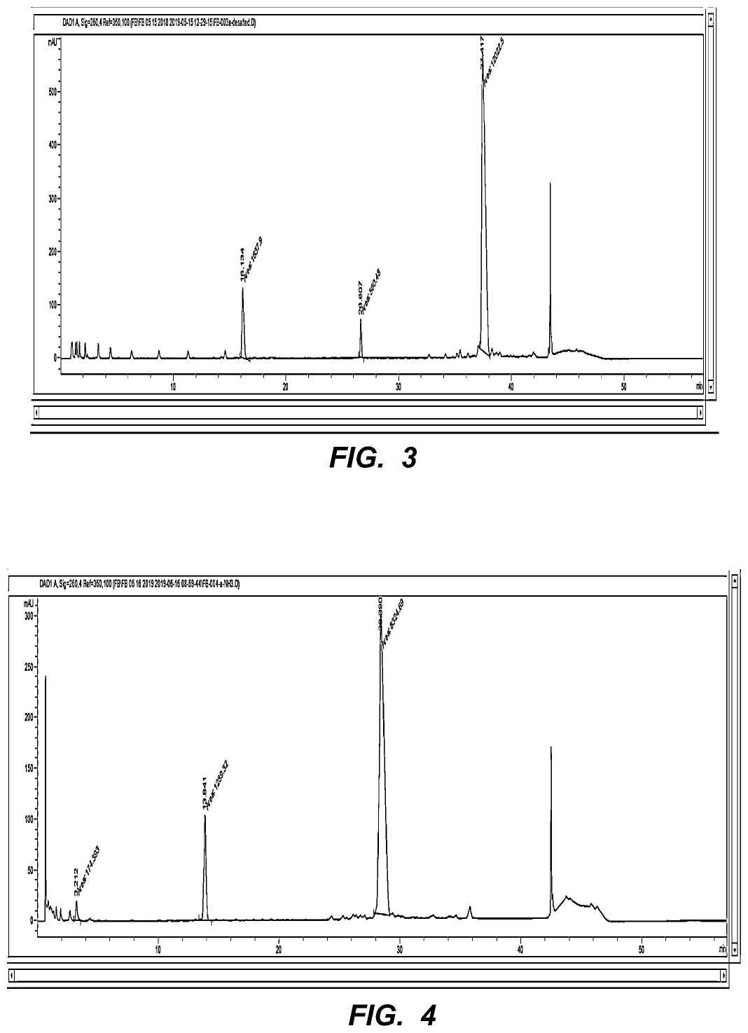

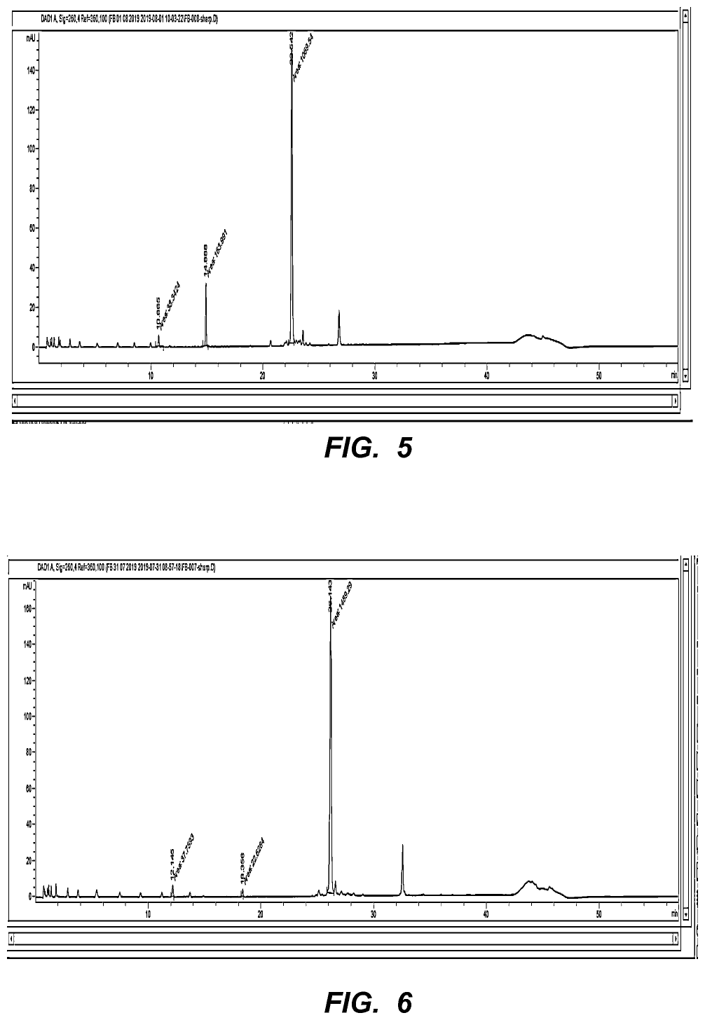

### DETAILED DESCRIPTION

In one aspect, the disclosure provides an improved method for preparing oligonucleotides comprising at least one nucleoside having a 3′-hydroxyl group. A nucleoside phosphoramidite monomer comprising a triisopropylsilylether (TIPS) protected 3′-hydroxyl group is coupled to a free hydroxyl, e.g., 5′-OH, 3′-OH or 2′-OH, preferably a 5′-OH, on a nucleoside or an oligonucleotide.

Methods and reagents for coupling nucleoside phosphoramidite monomers to hydroxyl groups are well known in the art. Thus, the oligonucleotide can be prepared using procedures and equipment known to those skilled in the art. For example, a glass reactor such as a flask can be suitably employed. Preferably, solid phase synthesis procedures are employed, and a solid support such as controlled pore glass. Even more preferably, the methods of the present invention can be carried out using automatic DNA synthesizers. Suitable solid phase techniques, including automated synthesis techniques, are described in F. Eckstein (ed.), Oligonucleotides and Analogues, a Practical Approach, Oxford University Press, New York (1991).

In addition, the oligonucleotide can be prepared in small scale or large scale. For example, the oligonucleotide can be prepared in the μmol scale or mg scale.

The coupling step and the oxidation/sulfurization step can be performed in a common solvent. For example, coupling and oxidation/sulfurization can be performed in acetonitrile.

Oxidation step can be carried out by contacting the phosphite triester intermediate with an oxidation reagent for a time sufficient to effect formation of a phosphotriester functional group. Suitable solvent systems for use in the oxidation of the phosphite intermediate of the present invention include mixtures of two or more solvents. Preferably a mixture of an aprotic solvent with a protic or basic solvent. Preferred solvent mixtures include mixtures of acetonitrile with a weak base. For example, the oxidation step can be carried out in presence of a weak base. Exemplary bases include, but are not limited to, pyridine, lutidine, picoline or collidine. In some embodiments, the oxidation step can be carried out in presence of I₂/H₂O.

Sulfurization (oxidation utilizing a sulfur transfer reagent) can be carried out by contacting the phosphite triester intermediate with a sulfur transfer reagent for a time sufficient to effect formation of a phosphorothioate functional group. Exemplary sulfur transfer reagents for use in oligonucleotide synthesis include, but are not limited to, phenylacetyl disulfide, arylacetyl disulfide, and aryl substituted phenylacetyl disulfides. For example, the sulfur transfer reagent can be 3-(dimethylaminomethylidene)amino-3H-1,2,4-dithiazole-3-thione (DDTT) or 3H-1,2-benzodithiol-3-one 1,1-dioxide (Beaucage reagent).

After synthesis is complete, the oligonucleotide can be deprotected, e.g., using methods and reagents to remove any protecting groups on the oligonucleotide to obtain the desired product. Accordingly, in some embodiments, the method further comprises treating the synthesized oligonucleotide with a base to remove any non-TIPS protecting groups on the oligonucleotide. Exemplary bases for use in removing non-TIPS protecting groups used in oligonucleotide synthesis include, but are not limited to, ammonium hydroxide, methylamine, and mixtures thereof. Treating with the base can suitably be carried out at room temperature or elevated temperature. “Room temperature” includes ambient temperatures from about 20° C. to about 30° C. “Elevated temperature” includes temperatures higher than 30° C. For example, elevated temperature can a temperature between about 32° C. to about 65° C. In some embodiments, treatment with the base is at about 35° C. The treatment times are on the order of few minutes, such as, for example 5, 10, 15, 20, 25, 30, 45 or 60 minutes, to hours, such as, for example, 2 hours, 3 hours, 4 hours, 5 hours, 10 hours, 15 hours 24 hours or longer. In some embodiments, treatment with the base is for about 15 hours. In some embodiments, treatment with the base is at about 35° C. for about 15 hours.

After the non-TIPS protecting groups have been removed, the TIPS protecting group can be removed by treating the partially deprotected oligonucleotide with a deprotecting reagent effective to convert the TIPS-protected hydroxyl group to a free hydroxyl group. Methods and reagents for removing silyl containing hydroxyl protecting groups are well known in the art. Generally, the deprotecting reagent comprises fluoride anions. One exemplary deprotecting reagent for removing TIPS protecting group is HF·pyridine. The deprotecting step for removing the TIPS groups can suitably be carried out at room temperature or elevated temperature. For example, the deprotection step can be carried out a temperate of between 35° C. to about 65° C. IN some embodiments, the deprotection step is carried out at around 50° C. The deprotection times are on the order of few minutes, such as, for example 5, 10, 15, 20, 25, 30, 45 or 60 minutes, to hours, such as, for example, 2 hours, 3 hours, 4 hours or 5 hours. In some embodiments, the oligonucleotide is treated with the deprotecting reagent for about 1 hour.

After deprotection, the desired product can be isolated and purified using method known in the art for isolation and purification of oligonucleotide. Such methods include, but are not limited to, filtration and/or HPLC purification.

In another aspect, the disclosure provides nucleoside monomers having a triisopropylsilylether (TIPS) protected 3′-hydroxyl group, e.g., monomer having the structure of Formula (I):

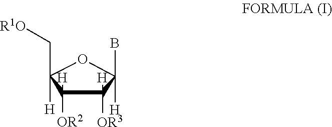

In monomers of Formula (I), B is a modified or unmodified nucleobase. Optionally, the nucleobase can comprise one or more protecting groups. Exemplary nucleobases include, but are not limited to, adenine, guanine, cytosine, uracil, thymine, inosine, xanthine, hypoxanthine, nubularine, isoguanisine, tubercidine, and substituted or modified analogs of adenine, guanine, cytosine and uracil, such as 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613.

In some embodiments, nucleobase can be selected from the group consisting of adenine, guanine, cytosine, uracil, thymine, inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyl)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶,N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N4-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N3-(methyl)uracil, 5-uracil (i.e., pseudouracil), 2-(thio)pseudouracil, 4-(thio)pseudouracil, 2,4-(dithio)pseudouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2 (thio)-pseudouracil, 1-substituted 4-(thio)pseudouracil, 1-(aminocarbonylethylenyl)-pseudouracil, 1-substituted 2,4-(dithio)pseudouracil, 1-(aminocarbonylethylenyl)-2 (thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2 (thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N2-substituted purines, N6-substituted purines, 06-substituted purines, substituted 1,2,4-triazoles, and any O-alkylated or N-alkylated derivatives thereof. In some embodiments, the nucleobase is selected from the group consisting of adenine, guanine, cytosine and uracil.

R¹ is a hydroxyl protecting group. The protecting group conventionally used for the protection of nucleoside 5′-hydroxyls is 4,4′-dimethoxytrityl (“DMT”). However, any hydroxyl protecting group known and used in the art for oligonucleotide synthesis can be used. Such protecting groups include, but are not limited to, monomethoxytrityl (“MMT”), 9-fluorenylmethylcarbonate (“Fmoc”), o-nitrophenylcarbonyl, p-phenylazophenylcarbonyl, phenylcarbonyl, p-chlorophenylcarbonyl, and 5′-(α-methyl-2-nitropiperonyl)oxycarbonyl (“MeNPOC”). Preferably, R¹ is an acid labile hydroxyl protecting group, e.g., DMT or MMT. In some embodiments, R1 is DMT.

Each R⁴ can be selected independently from the group consisting of alkyl, aryl, aralkyl, alkaryl, cycloalkyl, alkenyl, cycloalkenyl, alkynyl or cycloalkynyl, each of which can be optionally substituted, for example with 1, 2, 3, 4 or more independently selected substituents. For example, each R⁴ can be independently an optionally substituted C₁-C₆alkyl. Exemplary alkyls for R⁴ include, but are not limited to methyl, ethyl, propyl, isopropyl, butyl, 2-methylpropyl, t-butyl, and pentyl. In some embodiments, each R⁴ is isopropyl.

R³ can be H or —P(NR⁵R⁶)OR⁷. In some embodiments, R³ is H. In some other embodiments, R³ is —P(NR⁵R⁶)OR⁷. When R³ is —P(NR⁵R⁶)OR⁷, R⁵ and R⁶ can be selected independently from the group consisting of alkyl, aryl, aralkyl, alkaryl, cycloalkyl, alkenyl, cycloalkenyl, alkynyl and cycloalkynyl, each of which can be optionally substituted, for example with 1, 2, 3, 4 or more independently selected substituents, or R⁵ and R⁶ can be linked to form a heterocyclyl, which can be optionally substituted, for example with 1, 2, 3, 4 or more independently selected substituents. For example, R⁵ and R⁶ can be independently an optionally substituted C₁-C₆alkyl. Exemplary alkyls for R⁵ and R⁶ include, but are not limited to methyl, ethyl, propyl, isopropyl, butyl, 2-methylpropuyl, t-butyl, and pentyl. In some embodiments, R⁵ and R⁶ are isopropyl.

R⁷ is alkyl, aryl, aralkyl, alkaryl, cycloalkyl, alkenyl, cycloalkenyl, alkynyl or cycloalkynyl, each of which can be optionally substituted, for example with 1, 2, 3, 4 or more independently selected substituents. For example, each R⁷ can be independently an optionally substituted C₁-C₆alkyl. Exemplary alkyls for R⁷ include, but are not limited to, optionally substituted methyl, ethyl, propyl, isopropyl, butyl, 2-methylpropuyl, t-butyl, and pentyl. In some embodiments, R⁷ is β-cyanoethyl.

In some embodiments of monomers of Formula (I), B is adenine, guanine, cytosine, thymine or uracil; R¹ is monomethoxytrityl or dimethoxytrityl; R⁴ are independently optionally substituted C₁-C₆alkyl; and R³ is H and R⁷ is an optionally substituted C₁-C₆alkyl. For example, B is adenine, guanine, cytosine, thymine or uracil; R¹ is dimethoxytrityl; R⁴ are independently isopropyl; and R³ is H.

In some embodiments of monomers of Formula (I), B is adenine, guanine, cytosine, thymine or uracil; R¹ is monomethoxytrityl or dimethoxytrityl; R⁴ are independently optionally substituted C₁-C₆alkyl; R⁵ and R⁶ are independently optionally substituted C₁-C₆alkyl or R⁵ and R⁵ are linked to form a 4-8 membered heterocyclyl; and R⁷ is an optionally substituted C₁-C₆alkyl. For example, B is adenine, guanine, cytosine, uracil or thymine; R¹ is dimethoxytrityl; R⁴, R⁵ and R⁶ are isopropyl; and R⁷ is β-cyanoethyl.

Exemplary embodiments can be described by the following numbered embodiments:

Embodiment 1: A method for synthesizing oligonucleotides having at least one nucleoside with a 3′-OH group, the method comprising: (i) coupling a free hydroxyl group on a nucleoside or oligonucleotide with a nucleoside phosphoramidite monomer having a triisopropylsilylether (TIPS) protected 3′-hydroxyl group to form a phosphite triester intermediate; and (ii) oxidizing or sulfurizing said phosphite triester intermediate to form a protected intermediate.

Embodiment 2: The method of Embodiment 1, wherein all synthetic steps are performed on an automated oligonucleotide synthesizer.

Embodiment 3: The method of Embodiment 1 or 2, wherein oligonucleotide is synthesized at a large scale.

Embodiment 4: The method of any one of Embodiments 1-3, wherein said oxidizing is in presence of a weak base.

Embodiment 5: The method of Embodiment 4, wherein said weak base is pyridine, lutidine, picoline or collidine.

Embodiment 6: The method of any one of Embodiments 1-5, wherein said oxidizing is in presence of I₂/H₂O.

Embodiment 7: The method of any one of Embodiments 1-6, wherein said sulfurizing is in presence of a sulfur transfer reagent.

Embodiment 8: The method of Embodiment 7, wherein said sulfur transfer reagent is 3-(dimethylaminomethylidene)amino-3H-1,2,4-dithiazole-3-thione (DDTT) or 3H-1,2-benzodithiol-3-one 1,1-dioxide.

Embodiment 9: The method of any one of Embodiments 1-8, further comprising a step of deprotecting the protected intermediate with a base.

Embodiment 10: The method of Embodiment 9, wherein said base is ammonium hydroxide, methylamine, or a mixture of ammonium hydroxide and methylamine.

Embodiment 11: The method of Embodiment 9 or 10, wherein said treating with the base is at room temperature or an elevated temperature.

Embodiment 12: The method of any one of Embodiments 9-11, wherein said treating with the base is at a temperature of 30° C. or higher.

Embodiment 13: The method of any one of Embodiments 9-12, wherein said treating with the base is for at least 30 minutes.

Embodiment 14: The method of any one of Embodiments 9-13, wherein said treating with the base is for at least 4 hours.

Embodiment 15: The method of any one of Embodiments 9-14, further comprising treating the base treated intermediate with a deprotecting reagent effective to convert the TIPS-protected hydroxyl group to a free hydroxyl group

Embodiment 16: The method of Embodiment 15, wherein the deprotecting reagent comprises fluoride anions.

Embodiment 17: The method of Embodiment 15 or 16, wherein the deprotecting reagent is HF·pyridine.

Embodiment 18: The method of any one of Embodiments 15-17, wherein said treating with the deprotecting reagent is at temperature of 30° C. or higher.

Embodiment 19: The method of any one of Embodiments 1-18, wherein the oligonucleotide comprises from about 6 to about 50 nucleotides.

Embodiment 20: The method of any one of Embodiments 1-19, wherein the oligonucleotide comprises from about 10 to about 30 nucleotides.

Embodiment 21: A nucleoside monomer having the structure of Formula (I):

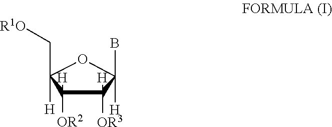

wherein B is a modified or unmodified nucleobase; R¹ is a hydroxyl protecting group; R² is —Si(R⁴)₃; R³ is H or —P(NR⁵R⁶)OR⁷; each R⁴ is independently optionally substituted alkyl, aryl, aralkyl, alkaryl, cycloalkyl, alkenyl, cycloalkenyl, alkynyl or cycloalkynyl; R⁵ and R⁶ are independently optionally substituted alkyl, aryl, aralkyl, alkaryl, cycloalkyl, alkenyl, cycloalkenyl, alkynyl or cycloalkynyl, or wherein R⁵ and R⁶ are linked to form a heterocyclyl; and R⁷ is optionally substituted alkyl, aryl, aralkyl, alkaryl, cycloalkyl, alkenyl, cycloalkenyl, alkynyl or cycloalkynyl.

Embodiment 22: The nucleoside monomer of Embodiment 21, wherein the hydroxyl protecting group is selected from the group consisting of 4,4′-dimethoxytrityl (DMT), monomethoxytrityl (MMT), 9-fluorenylmethylcarbonate (Fmoc), o-nitrophenylcarbonyl, p-phenylazophenylcarbonyl, phenylcarbonyl, p-chlorophenylcarbonyl, and 5′-(α-methyl-2-nitropiperonyl)oxycarbonyl (MeNPOC).

Embodiment 23: The nucleoside monomer of Embodiment 21 or 22, wherein each R⁴ is independently an optionally substituted C₁-C₆alkyl.

Embodiment 24: The nucleoside monomer of any one of Embodiments 21-23, wherein each R⁴ is isopropyl.

Embodiment 25: The nucleoside monomer of any one of Embodiments 21-24, wherein R⁵ and R⁶ are independently optionally substituted C₁-C₆alkyl.

Embodiment 26: The nucleoside monomer of any one of Embodiments 21-25, wherein R⁵ and R⁶ are isopropyl.

Embodiment 27: The nucleoside monomer of any one of Embodiments 21-26, wherein R⁷ is an optionally substituted C₁-C₆alkyl.

Embodiment 28: The nucleoside monomer of any one of Embodiments 21-27, wherein R⁷ is methyl or β-cyanoethyl.

Embodiment 29: The nucleoside monomer of any one of Embodiments 21-28, wherein B is adenine, guanine, cytosine, thymine or uracil; R¹ is monomethoxytrityl or dimethoxytrityl; R⁴ are independently optionally substituted C₁-C₆alkyl; R⁵ and R⁶ are independently optionally substituted C₁-C₆alkyl or R⁵ and R⁵ are linked to form a 4-8 membered heterocyclyl; and R⁷ is an optionally substituted C₁-C₆alkyl.

Embodiment 30: The nucleoside monomer of any one of Embodiments 1-29, wherein B is adenine, guanine, cytosine or uracil; R¹ is dimethoxytrityl; R⁴, R⁵ and R⁶ are isopropyl; and R⁷ is β-cyanoethyl.

### Some Selected Definitions

For convenience, certain terms employed herein, in the specification, examples and appended claims are collected herein. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

Further, the practice of the present invention can employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As used herein the term “comprising” or “comprises” is used in reference to compositions, methods, and respective component(s) thereof, that are essential to the invention, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “oligonucleotide” refers to a nucleic acid molecule (RNA or DNA) for example of length less than 100, 200, 300, or 400 nucleotides. As used herein, an oligonucleotide also encompasses dinucleotides, trinucleotides, tetranucleotides, pentanucleotides, hexanucleotides, and heptanucleotides. Further, the terms “nucleotide, nucleoside, oligonucleotide or an oligonucleoside” as used herein are intended to include both naturally occurring species and non-naturally occurring or modified species as is known to those skilled in the art.

The term “optionally substituted” means that the specified group or moiety is unsubstituted or is substituted with one or more (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

As used interchangeably herein, the terms “essentially” and “substantially” means a proportion of at least about 60%, or preferably at least about 70% or at least about 80%, or at least about 90%, at least about 95%, at least about 97% or at least about 99% or more, or any integer between 70% and 100%. In some embodiments, the term “essentially” means a proportion of at least about 90%, at least about 95%, at least about 98%, at least about 99% or more, or any integer between 90% and 100%. In some embodiments, the term “essentially” can include 100%.

As will be apparent to those of skill in the art upon reading this disclosure, each of the individual aspects described and illustrated herein has discrete components and features which can be readily separated from or combined with the features of any of the other several aspects without departing from the scope or spirit of the present invention. Any recited method can be carried out in the order of events recited or in any other order which is logically possible.

The invention is further illustrated by the following examples, which should not be construed as further limiting. The contents of all references, pending patent applications and published patents, cited throughout this application are hereby expressly incorporated by reference.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1: Synthesis of Phosphoramidites Having TIPS Protecting Group

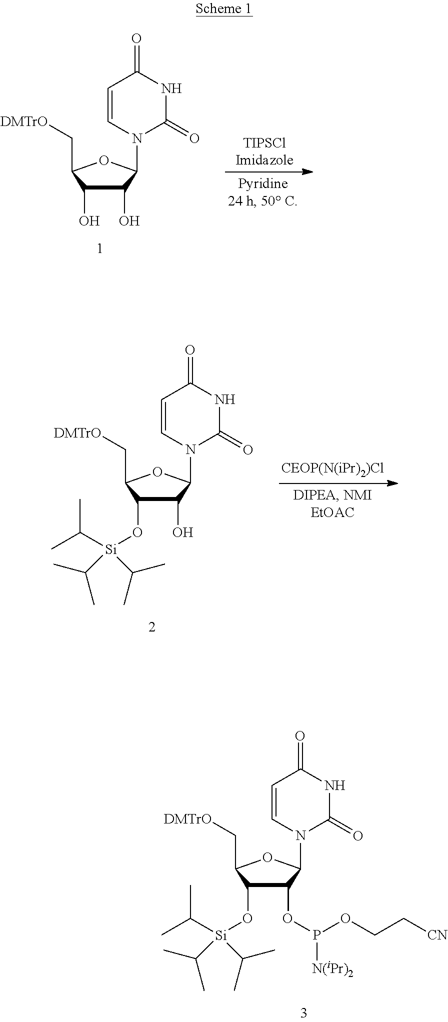

Compound 2: To a stirred solution of 5′-ODMTr uridine 1 (50 g, 91.48 mmol) in anhydrous pyridine (450 mL), imidazole (24.91 g, 365.92 mmol) and chloro(triisopropyl)silane (47.0 mL, 220 mmol) were added sequentially. After stirring for 24 h at 50° C., the volatiles were removed under reduced pressure. The residue was combined with an aqueous saturated solution of NaHCO₃ (400 mL) and EtOAc (500 mL), and stirred for 5 min. The mixture was transferred into a separatory funnel, the layers separated, and the organic layer was washed with an aqueous saturated solution of NaHCO₃, and brine. The organic layer was dried over Na₂SO₄, filtered and evaporated to dryness. The residue was purified by ISCO automated column. Dissolved in minimal DCM and loaded onto 120 g silica gel column using 0-30% EtOAc in hexanes as eluant to give compound 2 (26.1 g, 41%). ¹H NMR (500 MHZ, Acetonitrile-d3) δ 7.70 (d, J=8.2 Hz, 1H), 7.45-7.37 (m, 2H), 7.35-7.19 (m, 8H), 6.93-6.84 (m, 4H), 5.82 (d, J=3.9 Hz, 1H), 5.37 (d, J=8.1 Hz, 1H), 4.42 (t, J=5.4 Hz, 1H), 4.17 (td, J=5.3, 3.9 Hz, 1H), 3.77 (s, 6H), 3.49 (dd, J=10.9, 2.7 Hz, 1H), 3.31-3.23 (m, 2H), 1.06-0.90 (m, 22H). LRMS (ESI) calculated for C39H50N2O8Si [M+H]⁺ m/z=703.34. found 703.4.

Compound 3: DIPEA (19.3 mL, 111 mmol), 2-cyanoethyl-N,N-diisopropylchlorophosphoramidite (24.7 mL, 110.7 mmol), and N-methylimidazole (2.9 mL, 36.9 mmol) were added sequentially to a stirred solution of compound 2 (25.93 g, 36.89 mmol) in anhydrous EtOAc (600 mL) at 0° C. The cold bath was removed, and the reaction mixture was stirred for 1 h. The reaction was quenched with a solution of triethanolamine (2.7 M, 50 mL) in MeCN/toluene and stirred for 5 min. The mixture was diluted with ethyl acetate, transferred to a separatory funnel, layers separated, and the organic layer was washed sequentially with a 5% NaCl solution, and brine. The organic layer was dried over Na₂SO₄ and evaporated to dryness. The residue was pre-adsorbed on triethylamine pre-treated silica gel. The column was equilibrated with hexanes containing 1% NEt₃. The residue was purified by ISCO automated column using 0-40% EtOAc in hexanes as eluant to give compound 3 (26.5 g, 79%). ¹H NMR (500 MHZ, CD₃CN) δ 8.73 (s, 1H), 7.59 (d, J=8.1 Hz, 1H), 7.44-7.41 (m, 2H), 7.36-7.28 (m, 7H), 6.89-6.85 (m, 4H), 6.06 (d, J=5.4 Hz, 1H), 5.51 (d, J=8.1 Hz, 1H), 4.32-4.23 (m, 2H), 4.11-4.07 (m, 1H), 3.84-3.67 (m, 10H), 3.67-3.54 (m, 3H), 3.46 (dd, J=10.9, 3.7 Hz, 1H), 3.28 (dd, J=11.0, 4.2 Hz, 1H), 2.57 (t, J=6.2 Hz, 2H), 1.16-1.11 (m, 11H), 1.04-0.95 (m, 23H). ³¹P NMR (202 MHz, CD₃CN) δ 150.83, 150.80, 149.64, 149.61. LRMS (ESI) calculated for C48H67N4O9PSi [M+Na]⁺ m/z=902.44. found 925.2.

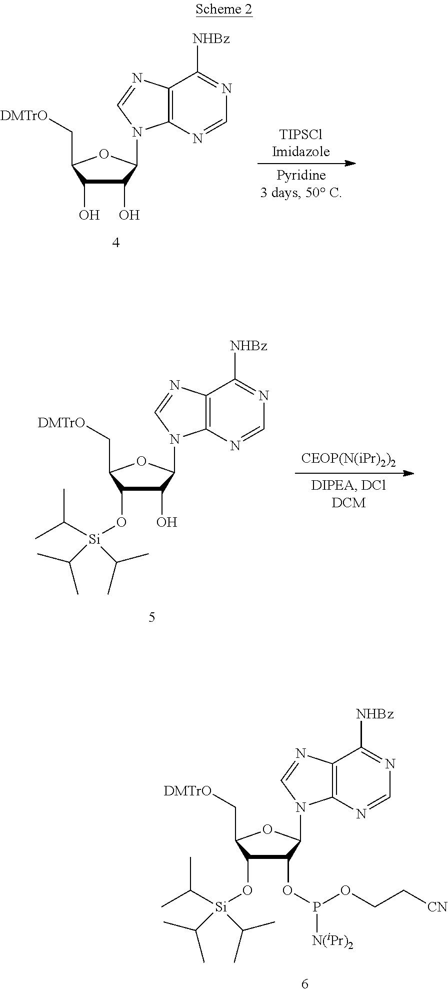

Compound 5: To a stirred solution of compound 4 (2.0 g, 3.0 mmol, 1 eq.) in anhydrous pyridine (15.0 mL), imidazole (1.62 g, 23.7 mmol, 8 eq.), and chloro(triisopropyl)silane (1.52 mL, 7.12 mol, 2.4 eq.) were added sequentially. After stirring for 24 h at 50° C., an aqueous saturated solution of NaHCO₃ (50 mL) and, Et₂O were added and the resulting mixture was transferred into a separatory funnel, the layers separated, and the aqueous layer was extracted with Et₂O (50 mL×2). The combined organic layer was dried over Na₂SO₄, filtered and evaporated to dryness. The residue was purified by ISCO automated column using 0-40% EtOAc in hexanes as eluant to give compound 5 (0.78 mg, 31%). ¹H NMR (500 MHZ, DMSO-d6) δ 11.22 (s, 1H), 8.64 (d, J=8.0 Hz, 2H), 8.08-8.02 (m, 2H), 7.67-7.61 (m, 1H), 7.57-7.52 (m, 2H), 7.39-7.32 (m, 2H), 7.28-7.16 (m, 8H), 6.88-6.80 (m, 4H), 6.06 (d, J=5.5 Hz, 1H), 5.50 (d, J=6.2 Hz, 1H), 4.96 (q, J=5.6 Hz, 1H), 4.65-4.59 (m, 1H), 4.15 (q, J=4.6 Hz, 1H), 3.72 (s, 6H), 3.41 (dd, J=10.5, 4.6 Hz, 1H), 3.20 (dd, J=10.5, 5.1 Hz, 1H), 1.14-0.93 (m, 24H). ¹³C NMR (101 MHz, DMSO) δ 166.15, 158.59, 152.51, 151.85, 151.01, 145.26, 144.51, 135.91, 135.88, 133.87, 132.92, 130.16, 128.98, 128.94, 128.22, 128.09, 127.15, 126.62, 113.60, 113.58, 88.78, 86.22, 84.61, 72.88, 72.65, 63.83, 55.51, 40.03, 18.34, 18.11, 18.01, 12.27. LRMS (ESI) calculated for C47H56N5O7Si [M+H]⁺ m/z=830.39. found 830.4.

Compound 6: To a stirred solution of compound 5 (201.5 g, 1.0 eq.) in anhydrous DCM (10 V), pyridine (6.0 eq), 2-Cyanoethyl N,N,N′,N′-tetraisopropylphosphorodiamidite (3.0 eq) and DCI (2.0 eq) were added. The mixture was stirring at 25° C. for 4 hours. After work up, the organic layer was dried over Na₂SO₄, filtered and evaporated to dryness. The reaction crude was precipitated with DCM/hept to give compound 6 (130 g, 52%). ³¹P NMR (202 MHZ, CDCl₃) δ 150.82, 150.66. LRMS (ESI) calculated for C56H73N7O8PSi [M+H]⁺ m/z=1031.49. found 1031.5.

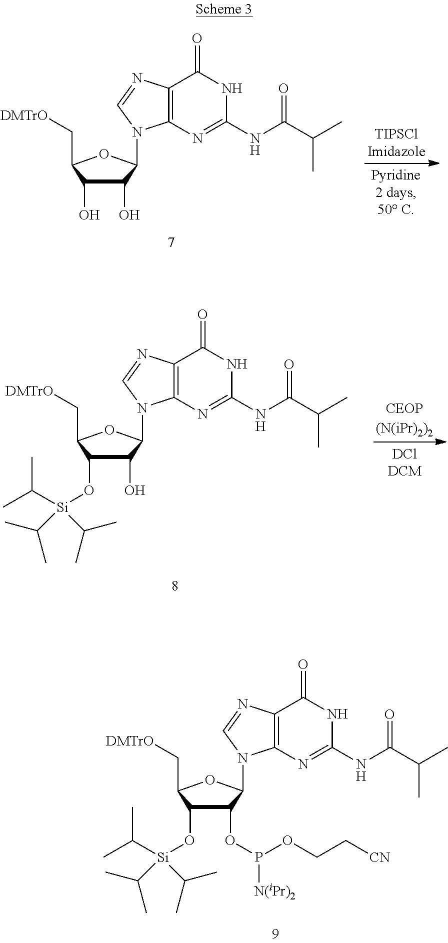

Compound 8: To a stirred solution of compound 7 (20.0 g, 30.5 mmol) in anhydrous pyridine (150.0 mL), imidazole (16.61 g, 0.24 mol) and chloro(triisopropyl)silane (26.1 mL, 0.12 mol) were added sequentially. After stirring for 24 h at 50° C., the volatiles were removed under reduced pressure. The residue was combined with an aqueous saturated solution of NaHCO₃ (100 mL) and EtOAc (500 mL), and stirred for 10 min. The mixture was transferred into a separatory funnel, the layers separated, and the organic layer was washed with an aqueous saturated solution of NaHCO₃ (50 mL) and brine (50 mL). The organic layer was dried over Na₂SO₄, filtered and evaporated to dryness. The residue was purified by ISCO automated column using 0-70% EtOAc in hexanes as eluant to give compound 8 (9.85 g, 40%). The column was equilibrated with hexanes containing 1% NEt₃. ¹H NMR (400 MHZ, CDCl₃) δ 11.96 (s, 1H), 7.88-7.81 (m, 1H), 7.54-7.48 (m, 1H), 7.42-7.36 (m, 1H), 7.30-7.18 (m, 1H), 6.85-6.76 (m, 1H), 5.70 (d, J=6.3 Hz, 1H), 4.95-4.88 (m, 1H), 4.62-4.57 (m, 1H), 4.54-4.50 (m, 1H), 4.17-4.13 (m, 1H), 3.77 (d, J=3.5 Hz, 9H), 3.59-3.52 (m, 1H), 3.27-3.16 (m, 1H), 3.14-3.05 (m, 1H), 1.72-1.62 (m, 1H), 1.33-1.20 (m, 1H), 1.01-0.88 (m, 1H), 0.72 (d, J=6.9 Hz, 4H), 0.60-0.45 (m, 3H). LRMS (ESI) calculated for C44H57N5O8Si [M+H]⁺ m/z=811.40. found 812.2.

Compound 9: To a stirred solution of compound 8 (140 g, 1.0 eq.) in anhydrous DCM (1.4 L), 2-Cyanoethyl N,N,N′,N′-tetraisopropylphosphorodiamidite (5.0 eq) and DCI (3.0 eq) were added. The mixture was stirring at 25° C. for 12 hours. The reaction was washed with 10% NaHCO₃ (10×1000 mL) and brine (2×1000 mL), dried over Na₂SO₄ and then concentrated at 35° C. to get crude product (387 g) as a light-yellow oil. The crude (386 g) was precipitated in DCM/MTBE several times (8 times) until compound 9 (81 g, 46%) was obtained as a white solid. ³¹P NMR (202 MHz, CDCl₃) δ 150.72, 149.33. LRMS (ESI) calculated for C53H75N7O9PSi [M+H]⁺ m/z=1012.5. found 1012.4.

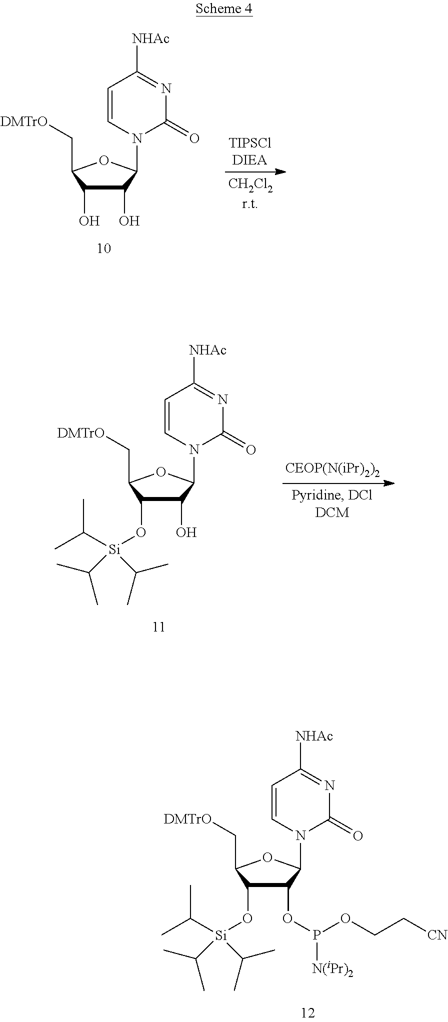

Compound 11: To a stirred solution of compound 10 (0.5 g, 0.85 mmol, 1 eq.) in anhydrous CH₂Cl₂ (2.8 mL), anhydrous diisopropylamine (0.72 mL, 5.1 mmol, 6 eq.) and chloro(triisopropyl)silane (0.55 mL, 2.5 mmol, 3 eq.) were added sequentially. After stirring at room temperature for 4 days, methanol (3 mL) was added and the resulting solution was stirred for 15 min. The mixture was diluted with DCM (10 mL) and the layer were separated. The organic layer was washed with water (10 mL×2) and dried over Na₂SO₄, filtered and evaporated to dryness. The residue was purified by ISCO automated column (the column was equilibrated with hexanes containing 1% NEt₃) using 0-60% EtOAc in hexanes as eluant to give compound 11 (287 mg, 45%). ¹H NMR (500 MHZ, DMSO-d6) δ 10.89 (s, 1H), 8.36 (d, J=7.5 Hz, 1H), 7.40-7.18 (m, 10H), 7.04 (d, J=7.5 Hz, 1H), 6.89 (dq, J=8.3, 3.2 Hz, 4H), 5.84 (d, J=2.5 Hz, 1H), 5.47 (d, J=5.7 Hz, 1H), 4.28 (dd, J=7.1, 4.8 Hz, 1H), 4.12-4.08 (m, 1H), 4.07-4.04 (m, 1H), 3.75 (d, J=0.8 Hz, 6H), 3.54 (dd, J=11.0, 2.9 Hz, 1H), 3.24 (dd, J=11.0, 3.8 Hz, 1H), 2.10 (s, 3H), 1.05-0.82 (m, 24H). ¹³C NMR (101 MHz, DMSO) δ 170.97, 170.30, 162.35, 158.24, 158.23, 154.47, 144.69, 144.19, 134.98, 134.93, 129.81, 129.78, 127.82, 126.91, 113.17, 113.13, 95.34, 91.03, 86.20, 82.34, 74.15, 70.29, 61.98, 59.73, 55.01, 39.52, 24.34, 20.74, 17.74, 14.07, 11.63. LRMS (ESI) calculated for C41H53N3O8SiNa [M+Na]⁺ m/z=766.35. found 766.3.

Compound 12: To a stirred solution of compound 11 (1.0 eq.) in anhydrous DCM (8 V), pyridine (6.5 eq), 2-Cyanoethyl N,N,N′,N′-tetraisopropylphosphorodiamidite (1.3 eq) and DCI (1.2 eq) were added. After stirring at 25° C. for 20 h, the mixture was washed with sat. NaHCO₃ and brine. After work up, the organic layer was concentrated to get crude compound 12 which was purified by column using 0-50% EtOAc in n-heptane containing 1% pyridine as eluent to give compound 12 (Yield: 76.6%). ³¹P NMR (202 MHz, CDCl₃) δ 151.96, 148.56. LRMS (ESI) calculated for C50H71N5O9PSi [M+H]⁺ m/z=944.4. found 944.1.

### Example 2: Synthesis of Uridine Having 3′-TOM and POM Protecting Groups

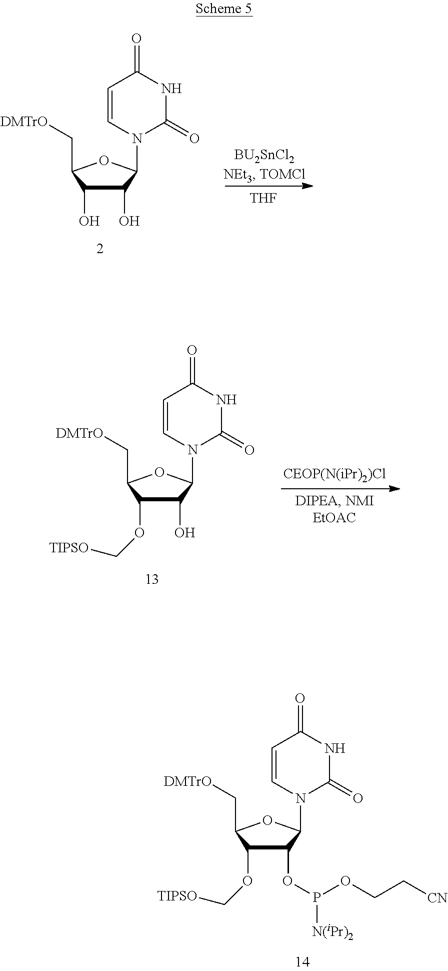

Compound 13: A solution containing of compound 2 (7 g, 13.1 mmol) and N-ethyl-N-isopropyl-propan-2-amine (8.01 mL, 46.01 mmol) in THF (50 mL) was treated with dibutyl(dichloro)stannane (4.58 g, 14.46 mmol, 3.36 mL) and stirred for 1 h at r.t. The reaction mixture was heated to 66° C., followed by addition of chloromethoxy (triisopropyl)silane (4.13 g, 15.77 mmol, 4.31 mL), and stirred for 40 min at 66° C. The reaction mixture was cooled to room temperature, and the volatiles were removed under reduced pressure. The crude residue was partitioned between DCM and a sat. solution of NaHCO₃, the layers were separated, and the organic layer was washed with an aqueous solution of NaHCO₃, brine, and dried over Na₂SO₄. The organic layer was dried over Na₂SO₄, filtered and evaporated to dryness. The residue was purified by ISCO automated column using 0-40% EtOAc in hexanes as eluant to give compound 13 (3.48 g, 37%). ¹H NMR (400 MHZ, CDCl₃) δ 7.77 (d, J=8.2 Hz, 1H), 7.39-7.22 (m, 1H), 6.87-6.80 (m, 1H), 5.96 (d, J=4.4 Hz, 1H), 5.39 (d, J=8.1 Hz, 1H), 5.06 (d, J=4.9 Hz, 1H), 4.90 (d, J=4.9 Hz, 1H), 4.35-4.22 (m, 1H), 3.80 (s, 6H), 3.59-3.51 (m, 1H), 3.43-3.36 (m, 1H), 2.05 (s, 2H), 1.60 (s, 2H), 1.13-1.01 (m, 2H). LRMS (ESI) calculated for C40H52N2O9Si [M+Na]⁺ m/z=732.34. found 755.4.

Compound 14: DIPEA (1.7 mL, 9.8 mmol), 2-cyanoethyl-N,N-diisopropylchlorophosphoramidite (2.2 mL, 9.81 mmol), and N-methylimidazole (0.39 mL, 4.9 mmol) were added sequentially to a stirred solution of compound 13 (3.5 g, 4.9 mmol) in anhydrous EtOAc (100 mL) at 0° C. The cold bath was removed, and the reaction mixture was stirred for 1 h. The reaction was quenched with a solution of triethanolamine (2.7 M, 11 mL) in MeCN/toluene and stirred for 5 min. The mixture was diluted with ethyl acetate, transferred to a separatory funnel, layers separated, and the organic layer was washed sequentially with a 5% NaCl solution, and brine. The organic layer was dried over Na₂SO₄ and evaporated to dryness. The residue was pre-adsorbed on triethylamine pre-treated silica gel. The column was equilibrated with hexanes containing 1% NEt₃. The residue was purified by ISCO automated column using 0-40% EtOAc in hexanes as eluant to give compound 14 (3.26 g, 71%). ¹H NMR (400 MHZ, CD₃CN) δ 7.69 (dd, J=9.7, 8.2 Hz, 1H), 7.46 (dd, J=7.2, 1.1 Hz, 2H), 7.36-7.21 (m, 7H), 6.90 (dd, J=7.6, 1.3 Hz, 4H), 6.00-5.96 (m, 1H), 5.43-5.35 (m, 1H), 5.12-4.96 (m, 2H), 4.56-4.48 (m, 1H), 4.42-4.36 (m, 1H), 4.33-4.25 (m, 1H), 3.91-3.58 (m, 11H), 3.47-3.33 (m, 2H), 2.68-2.61 (m, 2H), 1.25-0.94 (m, 36H). ³¹P NMR (162 MHz, CD₃CN) δ 150.61, 150.55. LRMS (ESI) calculated for C49H69N4O10PSi [M+H]⁺ m/z=932.45. found 955.5 (M+Na).

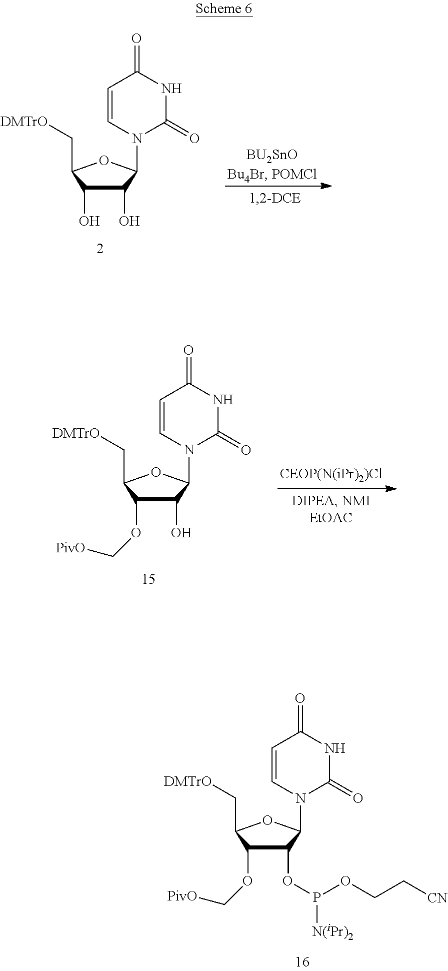

Compound 15: To an empty microwave tube, compound 2 (2 g, 3.76 mmol) was added, followed by addition of dibutyl(oxo)tin (1.22 g, 4.88 mmol, 769.23 uL) and tetrabutylammonium bromide (1.57 g, 4.88 mmol). The tube was closed with a rubber septum and the system was flushed with Ar for 5 minutes. 1,2-DCE (10 mL) was added and the resulting suspension was stirred for 1 min followed by addition of chloromethyl pivalate (1.41 g, 9.39 mmol, 1.35 mL). The septum was quickly exchanged for the microwave tube cap and the tube was heated in a microwave to 75° C. at 300 W for 2.5 h. Two more reactions with the same amount of reagents were done for a total of 6 g of compound 2. The three combined crude reaction mixture were combined and evaporated to dryness under reduced pressure. The sample was pre-adsorbed on silica pre-treated with triethylamine. The residue was purified by ISCO automated column (the silica was pre-treated with NEt₃) using 0-40% EtOAc in hexanes as eluant to give compound 15 (1.68 g, 23%). ¹H NMR (400 MHZ, CD₃OD) δ 7.87 (d, J=8.1 Hz, 1H), 7.48-7.36 (m, 3H), 7.35-7.22 (m, 4H), 6.94-6.84 (m, 2H), 5.89 (d, J=4.7 Hz, 1H), 5.41 (d, J=6.5 Hz, 1H), 5.37-5.27 (m, 1H), 4.50-4.38 (m, 2H), 4.23-4.17 (m, 1H), 3.54-3.39 (m, 1H), 3.35-3.28 (m, 1H), 1.20-1.08 (m, 4H). LRMS (ESI) calculated for C36H40N2O10 [M+H]⁺ m/z=660.27. found 661.7.

Compound 16: DIPEA (1.1 mL, 6.2 mmol), 2-cyanoethyl-N,N-diisopropylchlorophosphoramidite (1.4 mL, 6.2 mmol), and N-methylimidazole (0.19 mL, 2.4 mmol) were added sequentially to a stirred solution of compound 15 (1.6 g, 2.5 mmol) in anhydrous EtOAc (50 mL) at 0° C. The cold bath was removed and the reaction mixture was stirred for 1 h. The reaction was quenched with a solution of triethanolamine (2.7 M, 6 mL) in MeCN/toluene and stirred for 5 min. The mixture was diluted with ethyl acetate, transferred to a separatory funnel, layers separated, and the organic layer was washed sequentially with a 5% NaCl solution, and brine. The organic layer was dried over Na₂SO₄ and evaporated to dryness. The residue was pre-adsorbed on triethylamine pre-treated silica gel. The column was equilibrated with hexanes containing 1% NEt₃. The residue was purified by ISCO automated column using 0-60% EtOAc in hexanes as eluant to give compound 16 (1.517 g, 74%). ¹H NMR (500 MHZ, CD₃CN) δ 7.65-7.59 (m, 1H), 7.46-7.41 (m, 1H), 7.35-7.21 (m, 6H), 6.93-6.83 (m, 3H), 5.98-5.91 (m, 1H), 5.46-5.37 (m, 1H), 5.34 (d, J=6.5 Hz, 1H), 5.20 (d, J=6.4 Hz, 1H), 4.61-4.50 (m, 1H), 4.47-4.38 (m, 1H), 4.21-4.14 (m, 1H), 3.67-3.57 (m, 3H), 3.40-3.31 (m, 2H), 2.69-2.59 (m, 1H), 1.19-1.16 (m, 6H), 1.12 (t, J=6.4 Hz, 11H). ³¹P NMR (202 MHZ, CD₃CN) δ 150.84, 150.47.

### Example 3: Selective Synthesis of 3′-OTIPS Protected Nucleosides and Phosphoramidites

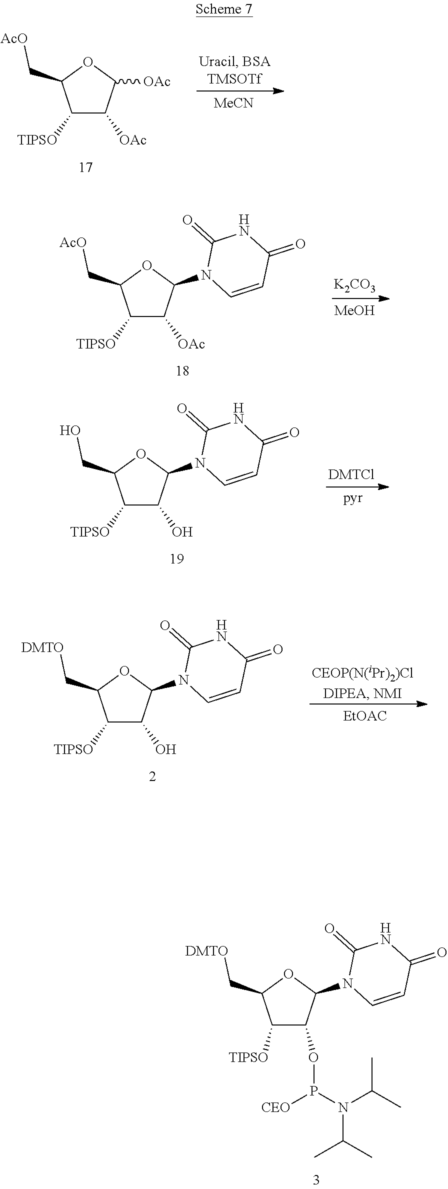

The synthesis started by installing the uracyl at the anomeric position of sugar 17 under Vorbrüggen conditions. The obtained compound 18 was treated with potassium carbonate to cleave the acetate groups producing nucleoside 19 which was protected at the 5′-O position with DMTCl to give nucleoside 2. Formation of the phosphoramidite 4 was achieved under standard conditions using 2-cyanoethyl-N,N-diisopropylchlorophosphoramidite.

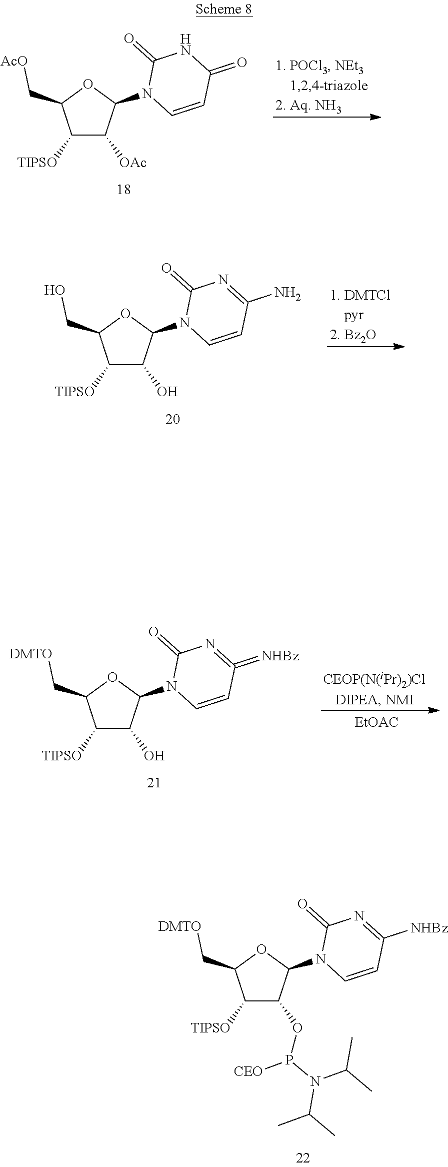

Starting from nucleoside 18, the uracyl nucleobase was transformed into a cytosine in a two-step triazolation/ammonolysis sequence to give nucleoside 20. Protection of the primary hydroxyl group with DMTCl and selective installation of a benzoate group at the nucleobase afforded nucleoside 21. Formation of the phosphoramidite 22 was achieved under standard conditions using 2-cyanoethyl-N,N-diisopropylchlorophosphoramidite.

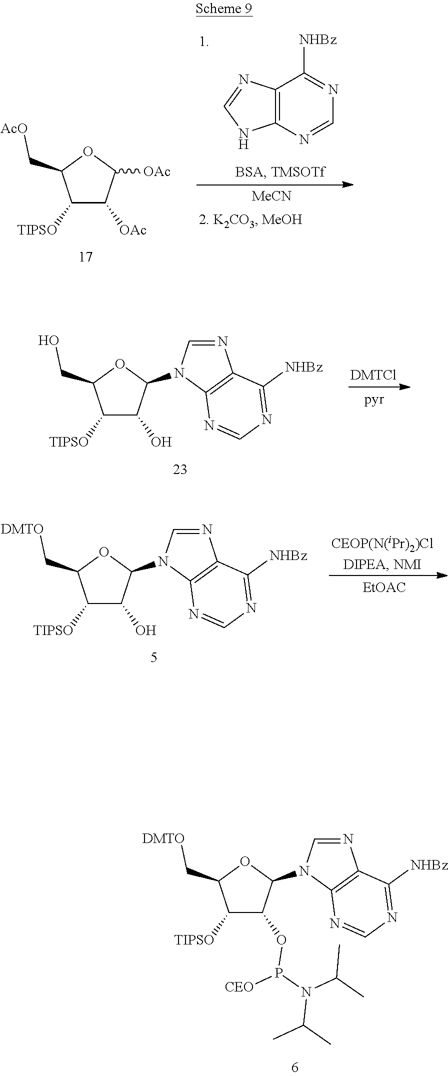

Transformation of sugar 17 into nucleoside 23 was achieved using N-benzoyl adenine under Vorbrüggen conditions followed by cleavage of the acetate groups under basic conditions. The primary hydroxyl in nucleoside 23 was protected as a DMT ether to give nucleoside 5 that was later transformer into the corresponding phosphoramidite 6 under standard conditions.

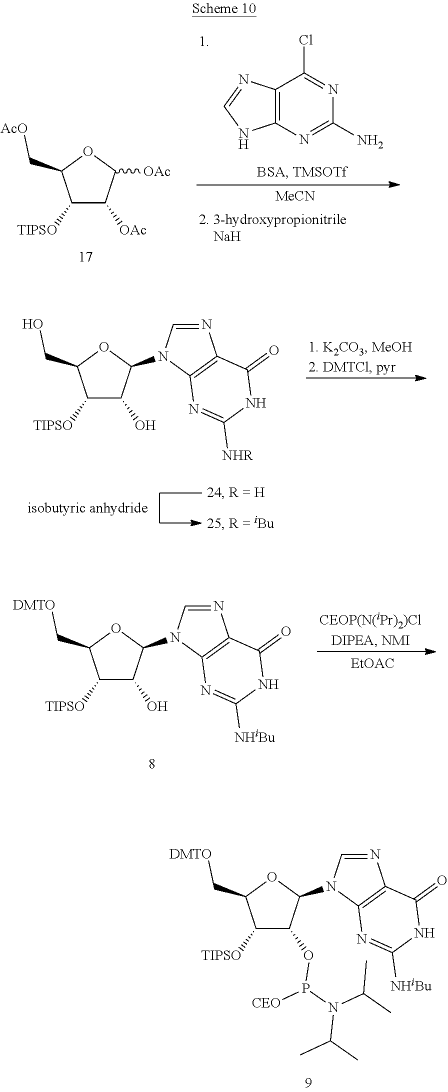

Using sugar 17 as starting material, nucleoside 24 was obtained using a two-step sequence to install the guanine moiety. The protection of the nucleobase with isobutyric anhydride gave compound 25. The acetate groups were cleaved under basic conditions and the primary hydroxyl group was protected as a DMT ether to give nucleoside 8. Formation of the phosphoramidite 9 was achieved under standard conditions using 2-cyanoethyl-N,N-diisopropylchlorophosphoramidite.

### Example 4. SiRNA Synthesis with 3′-O-Protected Nucleosides

Oligonucleotide synthesis: The synthesis of the representative oligonucleotides was performed using the parameters show in the tables below. The goal of this study was to determine the most optimal RNA protecting group that will be compatible with our current cleavage and deprotection methods (which involves prolonged exposure to aqueous base) and will minimize side reactions such as premature falling off protecting groups which may lead to RNA hydrolysis/cleavage. Conditions of synthesis are given in Tables 1 and 2, and the sequences of the synthesized oligonucleotides for these studies are summarized in Table 3.

TABLE 1

| | |
|---|---|
| Sequence | asCfsguuuXcaaagcAfcUfuuauusgsa (X = 3′-RNA) (SEQ |
| | ID NO: 11) |
| Scale | 1 μmol |
| Chemistry | 3′-RNA monomer on 17th position (denoted by letter “X”) |
| | from 3′, 23mer |
| Specific Parameters Applied | All amidites dissolved @ 100 mM in 100% MeCN |
| During Synthesis | or 85:15 MeCN:DMF; Activator @ 250 mM in |
| | 100% MeCN |
| | Coupling info all amidites: Amidite volume 80 μL |
| | (double coupled); Activator Volume 120 μL |
| | Activator:Amidite = 3.8:1 |
| Synthesis Equipment | MerMade 192 |
| Support | Universal Support 1000 Å |

TABLE 2

| | |
|---|---|
| Sequence | aUfcaaAfXCfAfcuuuAfuUfgaguuuc (X = 3′-RNA) (SEQ |
| | ID NO: 12) |
| Scale | 300 μmol |
| Chemistry | 3′-RNA monomer on 17th position (denoted by letter “X”) |
| | from 3′, 23mer |
| Specific Parameters Applied | Coupling info: Amidite volume 303 umol*2eq = 3.03 |
| During Synthesis | mL:Activator Volume: 8.08 mL |
| | Activator:Amidite = 4:1 |
| | For RNA monomer in X position: Used 2eq of |
| | amidite; coupling volume 3.03 mL |
| | Amidite Flow: 1.77 mL/min; Activator Flow: 4.72 |
| | mL/min |
| | 11 min recycling for all bases; all @ 12.88 mL/min |
| | Standard Final Detrit |
| | DEA Treatment at 75 cm/h for 20 min (12 CV) |
| Synthesis Equipment | AKTA Oligo Pilot Plus 100 using 12 mL column |
| Support | 2′-OMe-C(Ac) 600A CPG |

TABLE 3

| | | Exact | |
|---|---|---|---|
| SEQ | | Mass | |
| ID | | (with | Exact Mass (2′ |
| NO: | Sequence | PG*) | or 3′′free OH) |
| 1 | aUfcaaAf(U-2′-OTBS)CfAfcuuuAfuUfgaguuuc | 7565 | 7451 |
| 2 | aUfcaaAf(U-3′-OTBS)CfAfcuuuAfuUfgaguuuc | 7565 | 7451 |
| 3 | aUfcaaAf(G-3′-OTBS)CfAfcuuuAfuUfgaguuuc | 7605 | 7491 |
| 4 | aUfcaaAf(U-2′- | 7367 | 7451 |
| | OTOM)CfAfcuuuAfuUfgaguuuc | | |
| 5 | aUfcaaAf(U-3′- | 7367 | 7451 |
| | OTOM)CfAfcuuuAfuUfgaguuuc | | |
| 6 | aUfcaaAf(U-3′-OTIPS)CfAfcuuuAfuUfgaguuuc | 7607 | 7451 |
| 7 | CfAfcuuuAfuUfgaguuuc (3′-fragment) | | 5157 |
| 8 | asCfsguuu(U2p)caaagcAfcUfuuauusgsa | 7701 | 7587 |
| 9 | caaagcAfcUfuuauusgsa (3′-fragment) | | 5280 |
| 10 | asCfsguuu(U2p)P (5′-fragment) | | 2325 |
| 13 | asUfsgadAu(G2p- | 7773 | 7617 |
| | OTIPS)cugagaAfaUfacuccscsc | | |

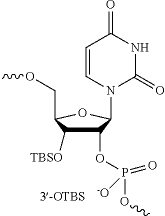

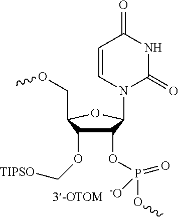

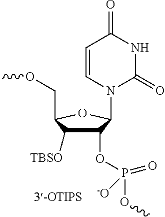

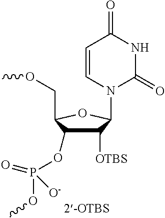

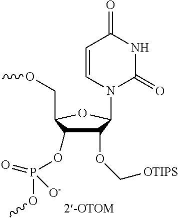

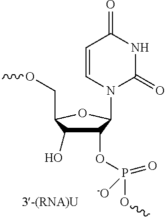

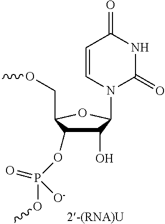

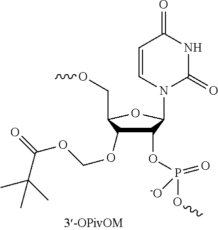

Cleavage and Deprotection: This deprotection is used to assess the quality of the synthesis, more specifically to identify impurities that are derived from premature deprotection of the RNA protecting group. Two different procedures were used depending on the scale of the synthesis (Procedure 1 for small scales and Procedure 2 for large scales). For both procedures NH₄OH, NH₄OH/EtOH, MeNH₂ or a mixture of ammonia/methylamine (AMA) can be used.

### Procedure 1:

1. After synthesis, the plate containing the columns was placed into a cleavage chuck over a 96-deepwell plate
2. Conc. aqueous methylamine solution or conc. ammonium hydroxide solution (150 μL) was added to each column and incubated for 30 mins at room temperature. The solution was subsequently drawn completely through the column using vacuum
3. Step #2 was repeated one more time, the plate sealed, and shaken at RT for the time specified.
4. A sample of the crude was diluted 100× with RODI water and analyzed using LCMS

### Procedure 2:

1. Small amount of the dried support (˜30 mg) after the synthesis is placed in a 2 mL glass screw cap vial.
2. Ammonium hydroxide solution (1 mL) was added and the vial was kept at 35° C. for 15 h. (Note: At this stage, the crude was cooled to room temperature then a sample was aliquoted, diluted 30× with RODI water then analyzed by HPLC for initial crude analysis)
3. For desilylation step: The crude solution was decanted, and the resin was washed 3 times with 0.5 mL DMSO. The vial was vortexed then left to stand for 2 minutes for all the resin to settle. The DMSO solution was decanted and was combined with initial filtrate into a 4 mL scintillation vial which was then cooled to 0° C. using ice bath.
4. Pyridine*HF (Sigma Aldrich, 0.75 mL) was added to the mixture (the reaction turned cloudy) and the vial was kept at 50° C. for 1 h.
5. The reaction was cooled to room temperature was quenched with water (2.5 mL). The vial was vortexed to dissolve all the solids.
6. A sample was aliquoted and diluted 30× with RODI water for HPLC analysis.

Analysis of crude oligonucleotide mixture by HPLC: Crude analysis was done using IPRP-LCMS using the conditions shown in Table 4.

TABLE 4

| | |
|---|---|
| Analytical column | ACQUITY UPLC Peptide BEH C18, 2.1 mm × 100 mm, 1.7 μm |
| Buffers/Solvents | Solvent A = 550 mM HFIP, 13 mM TEA, 10% MeOH, 5 μM |
| | EDTA |
| | Solvent B = MeOH, 5 μM EDTA |
| Working Gradient | Gradient A: 8-21% in 38 mins |
| (% B) | Gradient B: 5-27% in 38 mins |

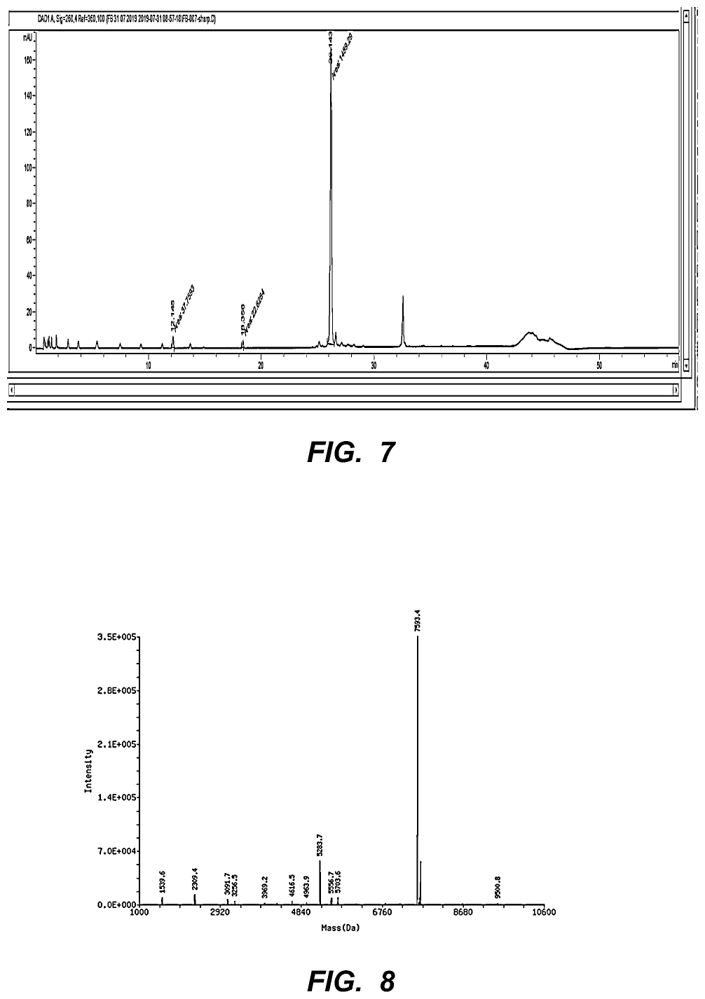

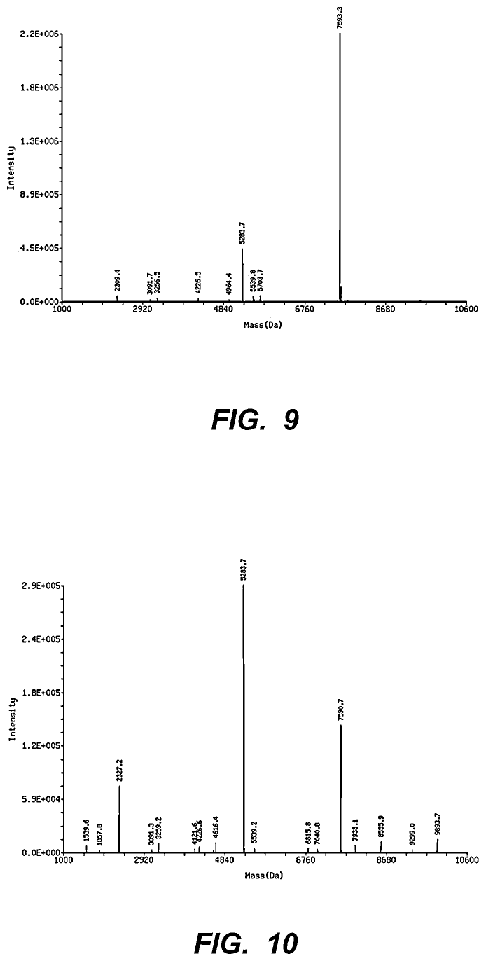

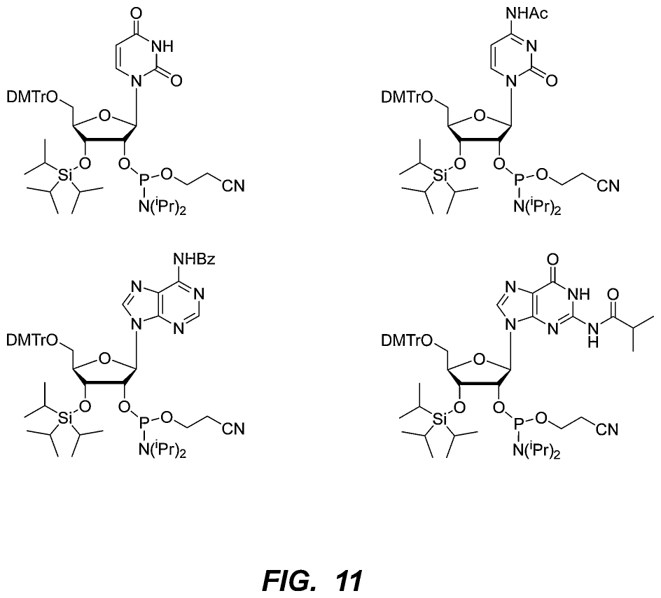

Results: Seven different 23mer oligonucleotides with different RNA protecting groups were synthesized (Table 3) and subjected to various cleavage and deprotection conditions. Where applicable, initial HPLC analysis was done prior to HF treatment to determine the stability of the various protecting groups during the base treatment. For simplicity, all HPLC and MS integrations were done only with the four species of interest; fully deprotected oligo having 3′ or 2′ hydroxyl group protected with silyl or other groups (FLP-OX−X=TBS, TOM, TIPS or Pivaloyloxymethyl), the deprotected oligo (FLP-OH), the cleaved 3′-fragment, and the cleaved 5′-fragment. As shown in Table 5, silyl protecting groups (TBS and TIPS) as well as TOM protecting group are unstable in prolonged base treatment, albeit to different degrees. The 23mer that contains the TIPS-protected RNA gave the best overall results with only 3% of the deprotected FLP and 1% of the cleaved hydrolyzed product. The protecting group (TIPS) can be easily removed using excess HF pyridine (FIG. 7) to generate FLP-OH. In addition, generation of and prolonged treatment of the FLP-OH to basic conditions can lead to varying levels of strand cleavage as shown in Table 5 and FIGS. 8-10.

TABLE 5

| RNA | Modification | C&D | | | % cleaved | % cleaved |
|---|---|---|---|---|---|---|
| Sequence* | (X) | condition | % FLP-OX** | % FLP-OH | (3′-fragment) | (5′-fragment) |
| 1 | 2′-OTBS-U | NH₄OH, | 87 | 9 | 4 | n.d. |
| | | 35° C., 15 h | | | | |
| 2 | 3′-OTBS-U | NH₄OH, | 87 | 2 | 11 | n.d. |
| | | 35° C., 15 h | | | | |
| 3 | 3′-OTBS-G | NH₄OH, | 84 | 4 | 12 | n.d. |
| | | 35° C., 15 h | | | | |
| 4 | 2′-OTOM-U | NH₄OH, | 84 | 14 | 2 | n.d. |
| | | 35° C., 15 h | | | | |
| 5 | 3′-OTOM-U | NH₄OH, | 84 | 13 | 3 | n.d. |
| | | 35° C., 15 h | | | | |
| 6 | 3′-OTIPS-U | NH₄OH, | 96 | 1 | 3 | n.d. |
| | | 35° C., 15 h | | | | |
| 8 | 3′-OPivOM-U | NH₄OH, | n.d. | 75 | 12 | n.d. |
| | | RT, 15 h | | | | |
| 8 | 3′-OPivOM-U | MeNH₂, | n.d. | 84 | 16 | n.d. |
| | | RT, 2 h | | | | |
| 8 | 3′-OPivOM-U | MeNH₂, | n.d. | 27 | 58 | 15 |
| | | RT, 15 h | | | | |
| 6 | 3′-OTIPS-U | NH₄OH, | 95 | 1 | 4 | n.d. |
| | | 35° C., 15 h | | | | |

*RNA sequence from Table 3.
**X = protecting groups on 3′ or 2′ (TBS, TOM, TIPS);
n.d. = none detected

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for synthesizing oligonucleotides having at least one nucleoside with a 3′-OH group, the method comprising:
(i) coupling a free 5′-hydroxyl group on a nucleoside or oligonucleotide with a nucleoside phosphoramidite monomer having (i-a) a 2′-phosphoramidite group and (i-b) a triisopropylsilylether (TIPS) protected 3′-hydroxyl group to form a phosphite triester intermediate;
(ii) oxidizing or sulfurizing said phosphite triester intermediate to form a protected intermediate; and
(iii) deprotecting the protected intermediate with a base, wherein said treating with the base is at a temperature of 30° C. or higher,
wherein the nucleoside phosphoramidite monomer has the structure of Formula (I): 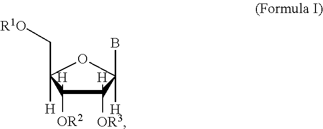
wherein:
B is a modified or unmodified nucleobase;
R¹ is a hydroxyl protecting group;
R² is —Si(R⁴)₃;
R³ is —P(NR⁵R⁶)OR⁷;
each R⁴ is independently isopropyl;
R⁵ and R⁶ are independently optionally substituted alkyl, or wherein R⁵ and R⁶ are linked to form a heterocyclyl; and
R⁷ is β-cyanoethyl.

2. The method of claim 1, wherein all synthetic steps are performed on an automated oligonucleotide synthesizer.

3. (canceled)

4. The method of claim 1, wherein said oxidizing is in presence of a weak base.

5. The method of claim 4, wherein said weak base is pyridine, lutidine, picoline or collidine.

6. The method of claim 1, wherein said oxidizing is in presence of I₂/H₂O.

7. The method of claim 1, wherein said sulfurizing is in presence of a sulfur transfer reagent.

8. The method of claim 7, wherein said sulfur transfer reagent is 3-(dimethylaminomethylidene)amino-3H-1,2,4-dithiazole-3-thione (DDTT) or 3H-1,2-benzodithiol-3-one 1,1-dioxide.

9. (canceled)

10. The method of claim 1, wherein said base is ammonium hydroxide, methylamine, or a mixture of ammonium hydroxide and methylamine.

11. The method of claim 1, wherein said treating with the base is at a temperature between 32° C. and 65° C.

12. The method of claim 11, wherein said treating with the base is at a temperature of 35° C.

13. The method of claim 1, wherein said treating with the base is for at least 30 minutes.

14. The method of claim 13, wherein said treating with the base is for at least 4 hours.

15. The method of claim 1, further comprising treating the base treated intermediate with a deprotecting reagent effective to convert the TIPS-protected hydroxyl group to a free hydroxyl group

16. The method of claim 15, wherein the deprotecting reagent comprises fluoride anions.

17. The method of claim 15, wherein the deprotecting reagent is HF-pyridine.

18. The method of claim 15, wherein said treating with the deprotecting reagent is at a temperature of 30° C. or higher.

19. The method of claim 1, wherein the oligonucleotide comprises from about 6 to about 50 nucleotides.

20. The method of claim 19, wherein the oligonucleotide comprises from about 10 to about 30 nucleotides.

21. A nucleoside monomer having the structure of Formula (I): 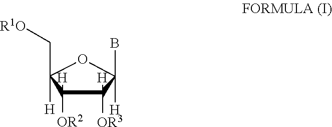
wherein:
B is a modified or unmodified nucleobase;
R¹ is a hydroxyl protecting group;
R² is —Si(R⁴)₃;
R³ is —P(NR⁵R⁶)OR⁷;
each R⁴ is isopropyl;
R⁵ and R⁶ are independently optionally substituted alkyl, or wherein R⁵ and R⁶ are linked to form a heterocyclyl; and
R⁷ is β-cyanoethyl.

22.-30. (canceled)
